(19) United States
(12) Patent Application Publication
(10) Pub. No.: US 2026/0232964 A1
(43) Pub. Date: Aug. 13, 2026

(54) Needles Including Coatings and Methods of Forming

(71) Applicant: COOK MEDICAL TECHNOLOGIES LLC, Bloomington, IN (US)

(72) Inventors: Weijin Qian, Kenmore (AU); John O'Mahony, Ormiston (AU); Tiffany Burleigh, Shailer Park (AU); Sharika Shalveen, Elanora (AU)

(21) Appl. No.: 19/538,460

(22) Filed: Feb. 12, 2026

Related U.S. Application Data
(60) Provisional application No. 63/757,897, filed on Feb. 13, 2025.

(51) Int. Cl. A61M 25/06; A61M 5/32

(52) U.S. Cl. CPC A61M 25/0631; A61M 5/3202; A61M 5/3216; A61M 25/0618; A61M 2005/3247

(57) ABSTRACT

A needle including a coating, medical device including the needle, and a method of coating the needle. The needle includes a metal cannula including an interior and exterior surface, and a coating disposed on at least one of the interior surface and the exterior surface. The coating is formed from a composition including a head group and a tail. The head group includes at least one of a thiol, a sulfide, a disulfide, a phosphonic acid, and a silane exhibiting the formula (—SiX₃). The tail includes at least one of an alkyl including in the range of 4 carbons to 18 carbons, a cyclic group including 6 carbons, a polycyclic group including 10 carbons, and a fluoroalkyl including in the range of 4 carbons to 18 carbons.

### BACKGROUND

The statements in this section merely provide background information related to the present disclosure and may not constitute prior art.

Needles are used in numerous medical and research applications. Such applications include tissue biopsy, therapeutics delivery by either intravenous or intramuscular routes, suturing, and oocyte retrieval, among many others. Various types of needles have been developed to address these applications including biopsy needles, intravenous catheter needles, cannula needles, hypodermic needles, dialysis needles, central venous catheter needles, and suturing needles. Needles may take on a number of configurations and form factors depending on the application for which they are used. In addition, needles are often designed with various characteristics in mind such as biocompatibility, ease of insertion, and minimization of tissue damage. For example, needles may exhibit varying external and internal diameters, lengths, and needle tip configurations. In addition, needles may be formed from a number of materials, including but not limited to stainless steel and nitinol.

During use needles often contact blood, medication, cellular debris, and other substances. These substances may adhere to needle surfaces and occlude needle openings. Clotting of blood, crystallization or aggregation of medication, and cellular debris, for example, may increase substance adherence to needle surfaces and occlusion of needle orifices and lumens. These issues may be exacerbated by needles being within the body for extended durations and by decreasing needle bore diameters. However, these issues may be addressed through various protocols, including flushing, use of anticoagulants, and altering medication formulation and packaging to reduce crystallization and aggregation.

Thus, while needles presently achieve their intended purpose, there is a need for new and improved needles that reduce the adhesion of substances to needle surfaces and occlusion.

### SUMMARY

According to various aspects, the present disclosure relates to a needle. The needle includes a metal cannula including an interior surface and an exterior surface. The needle also includes a coating disposed on at least one of the interior surface and the exterior surface, wherein the coating is formed from a composition including a head group and a tail. The head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine. The tail includes at least one of an alkyl including in the range of 4 carbons to 18 carbons, a cyclic group including 6 carbons, a polycyclic group including 10 carbons, and a fluoroalkyl including in the range of 4 carbons to 18 carbons.

In embodiments, the metal cannula includes at least one of stainless steel, titanium, nitinol, gold plated stainless steel, and cobalt-chromium.

In any of the above embodiments, the composition exhibits the formula (R—PO(OH)₂) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons. In further embodiments, the composition is octadecyl phosphonic acid. Alternatively, or additionally the composition is 3,3,4,4,5,5,6,6,7,7,8,8,8-tridecafluorooctylphosphonic acid.

Additionally, or alternatively, to the above embodiments, the composition exhibits the formula (R—S—S—R) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

Additionally, or alternatively, to the above embodiments, the composition exhibits the formula (R—SiX₃) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons, and X is at least one of the hydroxyl (—OH), the alkoxyl including in the range of 1 carbon to 6 carbons, and the halogen selected from at least one of chlorine, bromine, and iodine.

Additionally, or alternatively, to the above embodiments, the composition exhibits the formula (R—SH) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

Additionally, or alternatively, to the above embodiments, the composition exhibits the formula (R—S—R) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

According to various additional aspects, the present disclosure relates to a medical device. The medical device includes a needle according to any of the above embodiments. The needle includes a metal cannula including an interior surface and an exterior surface. The needle also includes a coating disposed on at least one of the interior surface and the exterior surface, wherein the coating is formed from a composition including a head group and a tail. The head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine. The tail includes at least one of an alkyl including in the range of 4 carbons to 18 carbons, a cyclic group including 6 carbons, a polycyclic group including 10 carbons, and a fluoroalkyl including in the range of 4 carbons to 18 carbons.

According to various further aspects, the present disclosure relates to a method of coating a needle. The method includes mixing a composition with a first medium to form a mixture, wherein the composition includes a head group and a tail. The head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine. The tail includes at least one of an alkyl including in the range of 4 carbons to 18 carbons, a cyclic group including 6 carbons, a polycyclic group including 10 carbons, and a fluoroalkyl including in the range of 4 carbons to 18 carbons. The method further includes immersing a needle including a metal cannula in the mixture, wherein the metal cannula includes an interior surface and an exterior surface and forming a coating on at least one of the interior surface and the exterior surface of the metal cannula.

In embodiments of the above, the first medium includes at least one of ethanol, isopropanol, n-butanol, n-propanol, methanol, acetone, chloroform, and tetrahydrofuran.

In any of the above embodiments, the composition is present in the first medium at a concentration on the order of 0.1 millimoles per liter to 10 millimoles per liter.

In any of the above embodiments, the metal cannula is immersed in the mixture for a time period on the order of 5 minutes to 48 hours.

In any of the above embodiments, the method also includes rinsing the metal cannula with a rinse after immersing the metal needle cannula. The rinse includes at least one of ethanol, isopropanol, n-butanol, n-propanol, methanol, acetone, chloroform, and tetrahydrofuran.

In any of the above embodiments, the method also includes drying the metal cannula including the coating at a temperature on the order of 100 degrees Celsius to 150 degrees Celsius.

In any of the above embodiments, the metal cannula is at least one of stainless steel, titanium, nitinol, gold plated stainless steel, and cobalt-chromium.

In any of the above embodiments, the composition exhibits the formula (R—PO(OH)₂) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

Additionally, or alternatively, in embodiments, the composition exhibits the formula (R—SiX₃) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons and X is at least one of the hydroxyl (—OH), the alkoxyl including in the range of 1 carbon to 6 carbons, and the halogen selected from at least one of chlorine, bromine, and iodine.

Additionally, or alternatively, in embodiments, the composition exhibits the formula (R—SH) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

### BRIEF DESCRIPTION OF DRAWINGS

The drawings described herein are for illustration purposes only and are not intended to limit the scope of the present disclosure in any way.

FIG. 1A illustrates a needle according to embodiments of the present disclosure.

FIG. 1B illustrates a cross-section of the needle of FIG. 1A taken along the length of the needle according to embodiments of the present disclosure.

FIG. 1C illustrates the needle of FIG. 1A provided in a sheath according to embodiments of the present disclosure.

FIG. 2 illustrates a coating on one of the exterior surface and the interior surface of a needle according to embodiments of the present disclosure.

FIG. 3 illustrates a measurement of water contact angle of a given surface in air.

FIG. 4 illustrates a method of forming a coating on a needle surface according to embodiments of the present disclosure.

FIG. 5 illustrates an ovum collection device according to embodiments of the present disclosure.

FIG. 6 illustrates a system for percutaneous access to an artery or vein according to embodiments of the present disclosure.

FIG. 7 illustrates a central venous catheter for use with a needle according to embodiments of the present disclosure.

FIG. 8 illustrates a biopsy device according to embodiments of the present disclosure.

FIG. 9 illustrates a therapeutic delivery system according to embodiments of the present disclosure.

FIG. 10 illustrates a hypodermic needle according to embodiments of the present disclosure.

FIG. 11 illustrates a blood collection needle according to embodiments of the present disclosure.

### DETAILED DESCRIPTION

The following description is merely exemplary in nature and is not intended to limit the present disclosure, application, or uses. Furthermore, there is no intention to be bound by any expressed or implied theory presented in the preceding introduction, summary, or the following detailed description. In context reference to “on the order of” is known to those skilled in the art and is used to indicate a reasonable range around a specific value and slight variations thereof, generally within a few percent of the values specified.

Reference will now be made in detail to several examples of the disclosure that are illustrated in accompanying drawings. Whenever possible, the same or similar reference numerals are used in the drawings and the description to refer to the same or like parts or steps. The drawings are in simplified form and are not to precise scale.

The present disclosure relates to needles that include a metal cannula with a coating disposed on the metal cannula, medical devices including the coated needles, and a method of applying a coating to metal cannulas of needles. While the present technology is described primarily herein in connection with needles used in oocyte retrieval, biopsy procedures, intravenous therapy and catheterization, dialysis, central venous catheter placement, blood collection, pain relief during childbirth or surgeries, spinal anesthesia, diagnostic lumbar punctures, minimally invasive procedures like laparoscopy and endoscopy, robotic surgery, diabetes management, hormone replacement therapy, embolization procedures, injection sclerotherapy, blood transfusions, dental procedures, acupuncture therapy, nerve stimulation therapy, intravitreal injection, radiofrequency ablation therapy, and hypodermic injections, the technology is not limited to these applications and may alternatively be employed in other applications, including applications where needles contact patients such as in insufflation and suturing, veterinary applications, as well as in applications where needles do not directly contact patients, such as in laboratory and research applications, including microfluidic devices, liquid dispensing, cell culture, bioprinting, electrospinning, and more.

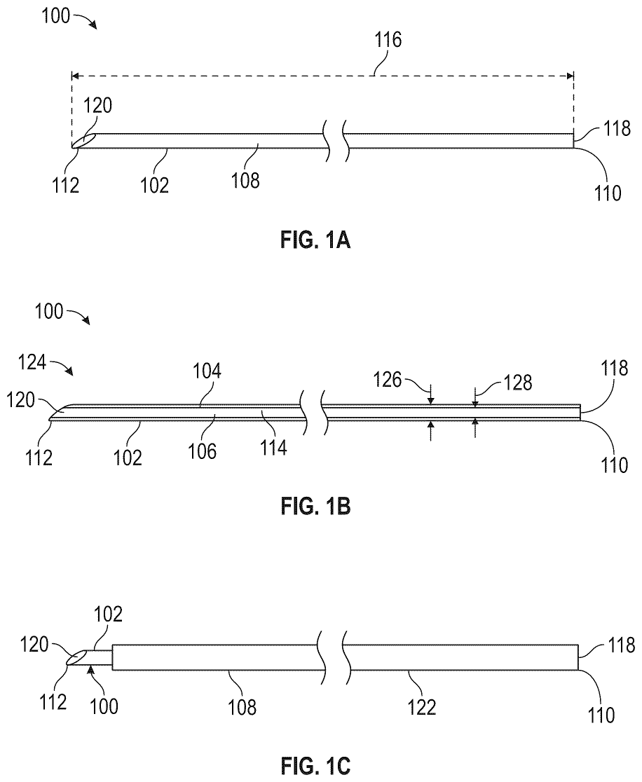

FIGS. 1A and 1B illustrate an embodiment of a needle 100, wherein FIG. 1B is a cross-section of the needle 100 of FIG. 1A. The needle 100 is generally cylindrical. In embodiments, the needle 100 includes a needle wall 104 that forms a cannula 102. The needle cannula 102 includes an interior surface 106 and an exterior surface 108. In addition, the cannula 102 includes a proximal end 110 and a distal end 112. The cannula 102 defines a bore 114 extending through the length 116 of the needle 100, which includes a proximal opening 118 defined at the proximal end 110 and a distal opening 120 defined at the distal end 112. In additional or alternative embodiments, one or more distal openings 120, providing one or more side ports, are located proximal to the distal end 112 and the distal end 112 may be either open or closed, depending on the application. In any of the above embodiments, the needle 100 includes a stylet. In alternative embodiments, the needle 100 is cylindrical in shape and does not include a bore 114 extending therethrough. In various applications, the needle 100 may be inserted into and through a sheath 122 as illustrated in FIG. 1C.

The cannula 102 is formed of metal. In embodiments, the metal includes at least one of stainless steel, titanium, nickel-titanium (i.e., nitinol), gold plated stainless steel, and cobalt-chromium. The stainless steel may include, but is not limited to, at least one of the following grades AISI 304, AISI 304L, AISI 316, AISI 316L, AISI 410, AISI 409, AISI 440B, AISI 440C, AISI 302, AISI 301, AISI 2205, AISI 904L, AISI 17-4 PH, AISI 17-7 PH, AISI 202, AISI 347, and other precipitation hardening stainless steels and austenitic stainless steels that do not have a direct AISI code such as 1RK91, and NANOFLEX available from ALLEIMA. In further embodiments, the needle 100 exhibits at least one of the following tip 124 styles: point style 2 (curve bevel), point style 3 (blunt or rounded), point style 4 (beveled at an angle selected from e.g., 12 degrees, 20 degrees and 45 degrees), point style 5 (conical needle with a side port), point style H (blunt including a side port), a point style including a bevel with two or three cutting edges, and point style AS (conical style, non-coring needle). The tip 124 of the cannula 102 is illustrated as being located at the distal end 112 of the needle 100. In embodiments, the proximal end 110 of the needle 100 may also include a tip (not illustrated) exhibiting one of the styles described above. In yet further embodiments, the cannula 102 exhibits a length 116 on the order of 5 millimeters to 600 millimeters, including all values and ranges therein, an outer diameter 126 on the order of 0.3 millimeters to 6 millimeters, including all values and ranges therein, and an inner diameter 128 on the order of 0.2 millimeters to 4 millimeters, including all values and ranges therein. The cannula 102 may also, in embodiments, include echo markings formed from dimples or other surface features formed on the exterior surface 108 of the needle 100. In addition, in embodiments, the metal forming the cannula 102 exhibits a surface energy of greater than 34 dyne per centimeter (dyne/cm), such as on the order of 34 dyne/cm to 53 dyne/cm, 70 dyne/cm to 1200 dyne/cm, including all values and ranges therein.

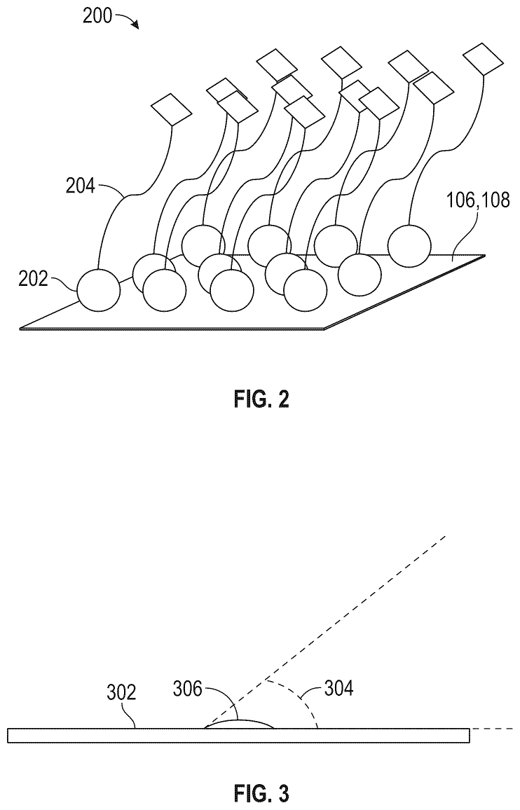

The cannula 102 includes a coating on at least one of the interior surface 106 and the exterior surface 108. In embodiments, the coating is provided on both the interior surface 106 and the exterior surface 108. The coating is disposed directly on, and in embodiments, bonded to the surfaces 106, 108. FIG. 2 illustrates the coating 200 on one of the interior surface 106 and exterior surface 108 of the cannula 102. In embodiments, the surface energy of coated needle surfaces 106, 108 is 70 dyne/cm or less, such as in on the order of 18 dyne/cm to 70 dyne/cm, including all values and ranges therein. In additional or alternative embodiments, the water contact angle of the coated surfaces 106, 108 is 60 degrees or above, such as on the order of 60 degrees to 120 degrees, including all values and ranges therein. The water contact angle, as described herein is the static water contact angle measured usually measured with a goniometer using the sessile-drop method, illustrated in FIG. 3, is measured on a surface 302 of the material of interest, such as the metal forming the cannula 102, whether it be uncoated or coated with the compositions described herein. A water bubble 306 is deposited on the surface 302 and the water contact angle 304 of the water bubble 306 is measured in air at temperatures in the range of 20 degrees Celsius to 30 degrees Celsius and a relative humidity of 30 percent to 70 percent.

The coating 200 is formed from a composition including a head group 202 and a tail 204. The head group 202 includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine. The tail 204 includes at least one of an alkyl including in the range of 4 carbons to 18 carbons, a cyclic group including 6 carbons, a polycyclic group including 10 carbons, and a fluoroalkyl including in the range of 4 carbons to 18 carbons. In embodiments, the coating 200 forms a self-assembled monolayer.

In embodiments the composition includes one or more phosphonic acids exhibiting the general formula (R—PO(OH)₂) wherein R is at least one of an alkyl including in the range of 4 carbons to 18 carbons, a cyclic group including 6 carbons, a polycyclic group including 10 carbons, and a fluoroalkyl including in the range of 4 carbons to 18 carbons. In further embodiments, the composition includes at least one of octadecylphosphonic acid, 3,3,4,4,5,5,6,6,7,7,8,8,8-tridecafluorooctylphosphonic acid, hexadecylphosphonic acid, n-dodecylphosphonic acid, octylphosphonic acid, tetradecylphosphonic acid, and decylphosphonic acid. In embodiments, the composition is 3,3,4,4,5,5,6,6,7,7,8,8,8-tridecafluorooctylphosphonic acid. In such embodiments, the surface energy of the coated surfaces 106, 108 may be 30 dyne/cm or less, such as on the order of 18 dyne/cm to 30 dyne/cm. Further, in such embodiments, the water contact angle may be 60 degrees or greater, such as on the order of 60 degrees to 120 degrees, including all values and ranges therein. In alternative embodiments, the composition is octadecyl phosphonic acid. In such embodiments, the surface energy of the coated surfaces 106, 108 may be 30 dyne/cm or less, such as on the order of 18 dyne/cm to 30 dyne/cm. Further, in such alternative embodiments, the water contact angle may be 80 degrees or greater, such as on the order of 80 degrees to 90 degrees, including all values and ranges therein.

Additionally or alternatively to the compositions above, the composition includes one or more disulfides exhibiting the general formula (R—S—S—R) wherein R is at least one of an alkyl including in the range of 4 carbons to 18 carbons, a cyclic group including 6 carbons, a polycyclic group including 10 carbons, and a fluoroalkyl including in the range of 4 carbons to 18 carbons. In embodiments, the composition includes octadecyl sulfide.

Additionally or alternatively to the compositions above, the composition includes one or more silanes exhibiting the formula (R—SiX₃) wherein R is at least one of an alkyl including in the range of 4 carbons to 18 carbons, a cyclic group including 6 carbons, a polycyclic group including 10 carbons, and a fluoroalkyl including in the range of 4 carbons to 18 carbons. In addition, X is at least one of a hydroxyl (—OH), an alkoxyl including in the range of 1 carbon to 6 carbons, and a halogen selected from at least one of chlorine, bromine, and iodine. In further embodiments, the composition includes at least one of trichloro (1H, 1H,2H,2H-perfluorooctyl) silane, 1H, 1H,2H,2H-perfluoro-octyltriethoxysilane, 1H,1H,2H,2H-perfluorododecyltrichloro-silane, and trichloro-(octadecyl) silane. In yet further embodiments, the composition includes trichloro (1H, 1H,2H,2H-perfluorooctyl) silane. In such further embodiments, the surface energy of the coated surfaces 106, 108 may be 30 dyne/cm or less, such as on the order of 18 dyne/cm to 30 dyne/cm.

Additionally or alternatively to the compositions above, the composition includes one or more thiols exhibiting the general formula (R—SH) wherein R is at least one of an alkyl including in the range of 4 carbons to 18 carbons, a cyclic group including 6 carbons, a polycyclic group including 10 carbons, and a fluoroalkyl including in the range of 4 carbons to 18 carbons. In further embodiments, the composition includes at least one of 1-octadecanethiol, 1-dodecanethiol, 1-hexadecanethiol, 1-tetradecanethiol, and 1-octanethiol. In embodiments, the composition is 1-octadecanethiol. In such further embodiments, the surface energy of the coated surfaces 106, 108 may be below 50 dyne/cm, such as on the order of 42 dyne/cm to 48 dyne/cm.

Additionally or alternatively to the compositions above, the composition includes one or more sulfides of the general formula (R—S—R) wherein R is at least one of an alkyl including in the range of 4 carbons to 18 carbons, a cyclic group including 6 carbons, a polycyclic group including 10 carbons, and a fluoroalkyl including in the range of 4 carbons to 18 carbons.

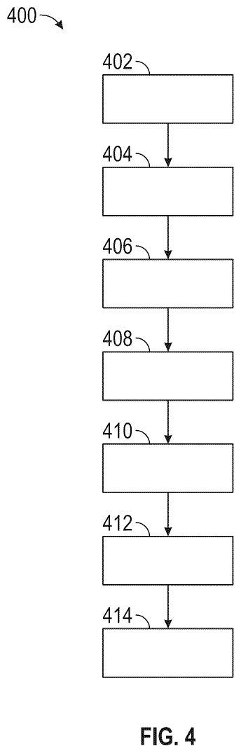

The coatings 300 are generally applied by immersion of the needles 100 in a mixture of at least one of the compositions described above and a first medium. FIG. 4, with further reference to FIGS. 1A through 3, illustrates an embodiment of a method 400 of coating a needle 100. It should be appreciated that more than one needle 100 may be coated at once. If not known already, at block 402, one or more calculations may be performed to determine the amount of the composition needed to obtain a desired concentration of the composition in a given amount of medium. An example of a calculation for determining the amount, or mass, of the composition for use in the medium is illustrated in EQ1, below.

$$Amt of Composition ( g ) = [ \text{} Total {Vol}_{medium} ( ml ) ] \times [ C ] \times [ \frac{1 {0}^{- 6} mol}{ml} ] \times [ MW ( \frac{g}{mol} ) ] \qquad EQ 1$$

wherein Amt of Composition is the amount of the composition, from one of the above described compositions, in grams (g), Total Vol_{medium} is the total volume of a first medium in milliliters (ml), C is the desired concentration in millimoles (mM), MW is the molecular weight of the composition in grams per mol (g/mol). As may be appreciated, the calculation may be performed using alternative equations and units of calculation. Further, more than one of the above noted compositions may be used and in such instances the calculations may be adjusted accordingly. In embodiments, the concentration of the composition, or the concentration of each composition if more than one composition is present, is on the order of 0.1 millimoles per liter (mM/L) to 10 mM/L, including all values and ranges therein, such as 0.5 mM/L, 1 mM/L, 2 mM/L, 5 mM/L, etc.

At block 404 a mixture of the composition and the first medium is prepared. The composition and the first medium are measured and added to a mixing container. The first medium includes an alcohol or other solvent having a molar mass on the order of 30 grams per mole to 120 grams per mole and a boiling point on the order of 50 degrees Celsius to 120 degrees Celsius. In embodiments, the first medium includes one or more alcohols with similar properties, such as short-chain alcohols or long-chain alcohols, including but not limited to ethanol, isopropanol, n-butanol, n-propanol, methanol or other suitable solvents including acetone, chloroform, tetrahydrofuran and mixtures thereof.

In embodiments, the container is covered, or otherwise enclosed, to reduce evaporation of the first medium during mixing. The composition is then mixed with the first medium until the composition is distributed in the medium. Mixing is performed for a time period on the order of 1 minute to 120 minutes, including all values and ranges therein, such as 5 minutes, 10 minutes, 30 minutes, 60 minutes, and 90 minutes. A magnetic stirrer or other mixer is used to mix the composition with the first medium. In embodiments, the composition becomes dissolved in the first medium to form a solution; otherwise, the mixture is a two-phase system. In further embodiments, the mixture exhibits a viscosity on the order of 0.1 to 10 centipoise, measured at 20° C. temperature. Prior to use, the mixture of the composition and first medium may be stored in a closed container.

In embodiments, before mixing, the mixing container and accessory tools, such as beakers, tweezers, syringes, Schott bottles, stirring bars, and pipettes, may be rinsed one or more times with a first rinse. The first rinse includes an alcohol or other solvent having a molar mass on the order of 30 grams per mole to 120 grams per mole and a boiling point on the order of 50 degrees Celsius to 120 degrees Celsius. In embodiments, the first rinse includes one or more alcohols with similar properties, such as short-chain alcohols or long-chain alcohols, including but not limited to ethanol, isopropanol, n-butanol, n-propanol, methanol or other suitable solvents including acetone, chloroform, tetrahydrofuran, and mixtures thereof. The first rinse may be, in embodiments, the same as the first medium. The composition and the first medium are then added to the rinsed mixing container and mixed as described above.

At block 406, one or more needles 100 are immersed in the mixture of the composition and the first medium. To prevent evaporation, in embodiments, the immersion container is covered with an airtight lid. The needles 100 are immersed for a time period on the order of 5 minutes to 48 hours, including all values and ranges therein. In embodiments, prior to immersing the needle 100, the needle 100 is cleaned and dried. In addition, any tools that are used during the immersion process are cleaned and dried. During immersion, a coating 300 is formed from the composition on at least one of the interior surface 106 and exterior surface 108 of the needle 100.

In further embodiments, if it is desired to coat only selected surfaces, surfaces that are to remain uncoated may be masked off. For example, if only the exterior surface 108 of a needle 100 is to be coated, the openings 118, 120 of the needle 100 may be plugged to prevent contact of the interior surface 106 with the mixture. Alternatively, if only the interior surface 106 of a needle 100 is to be coated or only portions of the exterior surface 108 is to be coated, the exterior surface 108 may be covered, such as by application of paraffin wax or other substance, to prevent contact of the exterior surface 108 with the mixture.

At block 408, the needles 100 are removed from the mixture. Agitation may be minimized to prevent or reduce disruption of the coating layer disposed on the needle surfaces. At block 410 the needles 100 are rinsed with a second rinse. The second rinse includes an alcohol or other solvent having a molar mass on the order of 30 grams per mole to 120 grams per mole and a boiling point on the order of 60 degrees Celsius to 120 degrees Celsius. In embodiments, the second rinse is one or more of alcohols with similar properties, such as short-chain alcohols or long-chain alcohols, including but not limited to ethanol, isopropanol, n-butanol, n-propanol, methanol or other suitable solvents including acetone, chloroform, tetrahydrofuran, and mixtures thereof. In embodiments, the second rinse is the same as the first medium. The needles 100 are then rinsed one or more times for a time period on the order of 10 to 15 seconds. Rinsing may remove any unbound composition.

At block 412 the needles 100 are dried. In embodiments, the needles 100 are dried in a drying oven for at least an hour, such as on the order of 1 hour to 10 hours, including all values and ranges therein, at a temperature on the order of 100 degrees Celsius to 150 degrees Celsius. At block 414 the needles 100 are conditioned at room temperature (21 degrees Celsius to 23 degrees Celsius, including all values and ranges therein) for a time period of at least 24 hours, including all values and ranges on the order of 24 hours to 48 hours.

In embodiments, the process may be repeated starting at block 406 and the needles 100 may be re-immersed in the mixture of the composition and the first medium one or more times, such as on the order of 2 to 20 times, or a mixture of another composition from those listed above and the first medium. Re-immersion may occur before the needles 100 are rinsed at block 410, after the needles 100 are rinsed at block 410 and before the needles 100 are dried at block 412, after the needles 100 are dried at block 412 and before conditioning at block 414, or after the needles 100 are conditioned at block 414.

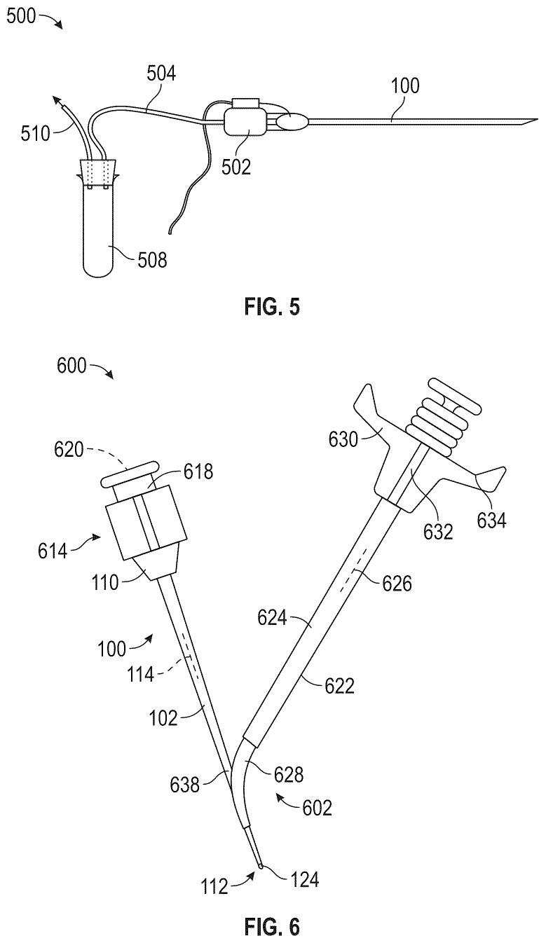

As noted above, the needles 100 including the coating described herein are used in a number of applications and medical devices for these applications. An example of an application includes an ovum collection device. FIG. 5 illustrates an embodiment of ovum collection device 500 for retrieval of oocytes. The device 500 includes a needle 100 including a coating as described herein extending from a handle 502. The needle 100 is connected to a tube 504, which provides a flow path from the needle 100 to a collection tube 508. An aspiration line 510 extends out of the collection tube 508. In embodiments, the collection tube 508 may include a warmer, which warms the collection tube 508 at a temperature on the order of 37 degrees Celsius.

Another example of an application for use with the needles 100 including the coating described herein is for percutaneous access to an artery or vein for intravenous therapy, dialysis, or catheterization. FIG. 6 illustrates an embodiment of a system 600 for percutaneous access to an artery or vein for intravenous therapy, dialysis, or catheterization. The system 600 includes a tubular medical device 602. A needle 100 including a coating as described herein passes through a portion of the tubular medical device 602. The needle 100, as illustrated, includes a cannula 102. The needle 100 also includes a beveled tip 124 at a distal end 112 of the needle 100. The cannula 102 defines a bore 114 that extends the length of the needle 100. A hub 614 can be affixed at the proximal end 110 of the needle 100. As illustrated, the hub 614 includes a fluid coupling 618, such as a Luer lock type connector. The hub 614 may define a passageway 620 for fluid to pass through the hub 614 and into the bore 114.

The system 600 may also include an introducer sheath 622. The introducer sheath 622 includes a sheath body 624 defines a lumen 626 through which a dilator 628 may be introduced. The sheath body 624 is coupled to a handle 630 including an opening 632 defined therein. The opening 632 provides a passageway into the sheath lumen 626. The handle 630 may include tabs 634, as illustrated, to support the fingers and/or thumb of the user. In embodiments, the dilator 628 includes a side port 638 to receive the needle 100.

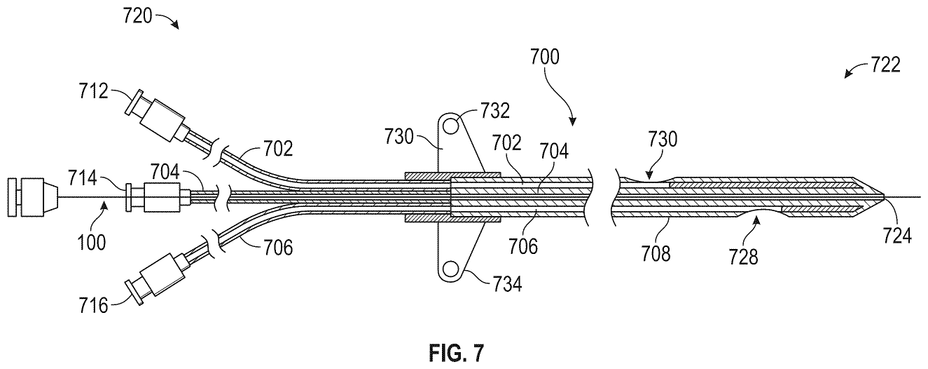

In additional applications, a needle 100 including a coating as described herein may also be used in a central venous catheter including multiple ports. FIG. 7 illustrates an embodiment of a central venous catheter 700 including a catheter body with a plurality of non-communicating lumens 702, 704, 706. While three lumens are illustrated, it should be appreciated that fewer than three lumens or more than three lumens, such as on the order of up to 6 lumens, may be present. The lumens 702, 704, 706 are defined in and extend through a body 708 of the catheter 700. Each lumen 702, 704, 706 includes an entry port 712, 714, 716 at a proximal end 720 of the catheter 700. At the distal end 722 of the catheter 700, the center lumen 704 includes an opening 724. Each side lumen 702, 706 includes a side opening 730, 728 near the distal end 722. A needle 100, such as the needle 100 illustrated in FIG. 6 may be inserted through the central lumen 704 and extend out of the opening 724 to create an opening for insertion of the catheter 700 into the vein providing access to the heart. The central venous catheter 700 may also include, as illustrated, a hub 730 from which wings 732, 734 protrude and can be used as anchor points for securing the catheter 700 in place.

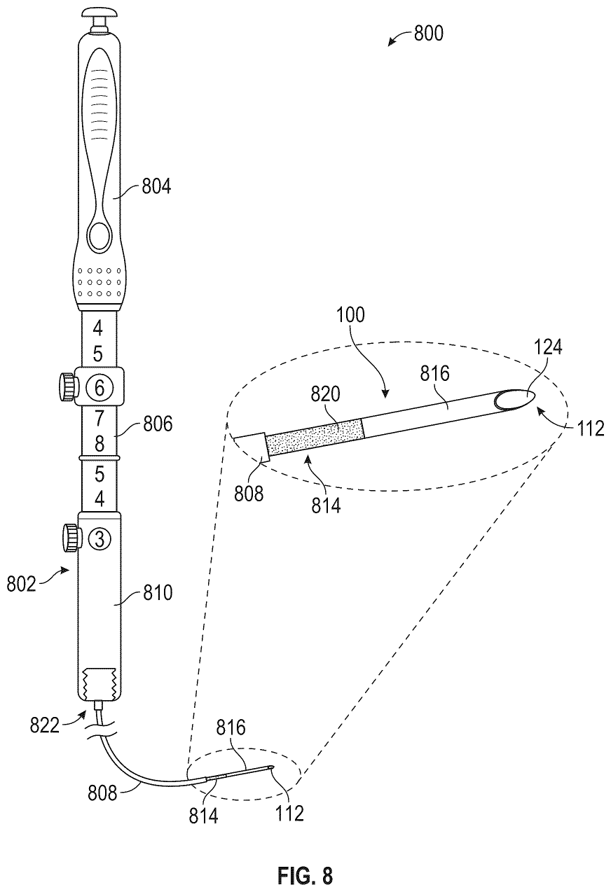

Turning now to FIG. 8, FIG. 8 illustrates an embodiment of a biopsy device 800 including a needle 100 including a coating as described herein. The biopsy device 800 includes a handle 802 with a handle member 804 attached to the biopsy needle 100. The handle member 804 is longitudinally slidable on a stem 806. The stem 806 is attached to a needle sheath 808. The needle 100 slides through the stem 806 and the needle sheath 808 when the handle member 804 is moved relative to the stem 806. The device 800, as illustrated, also includes an elongate scope mount 810. In embodiments, the stem 806 is slidable relative to the elongate scope mount 810. The needle 100 is extendable out of the distal end of the needle sheath 808. By moving the stem 806 distally, towards the elongate scope mount 810, causes the sheath 808 and needle 100 extend out, or extend out further, from the end 822 of the scope mount 810. Moving the stem 806 proximally, away from the elongate scope mount 810, causes the sheath 808 and needle 100 to retract partially, or completely, into the end 822 of the scope mount 810. Further, moving the handle member 804 distally over the stem 806 and towards the elongate scope mount 810 causes the needle 100 to advance distally from the sheath 808 and extend from, or extend further from, the distal end of the sheath 808. Moving the handle member 804 proximally over the stem 806 and away from the elongate scope mount 810 causes the needle to retract, at least partially, if not completely, into the sheath 808.

In the illustrated embodiment, the needle 100 includes two portions at the distal end 112, a proximal portion 814 and a distal portion 816. The distal portion 816 of the needle includes a beveled needle tip 124, however, other tip configurations may be used instead. At the proximal portion 814 of the distal end the illustrated embodiment includes surface features 820 detectable via ultrasound for locating the needle 100.

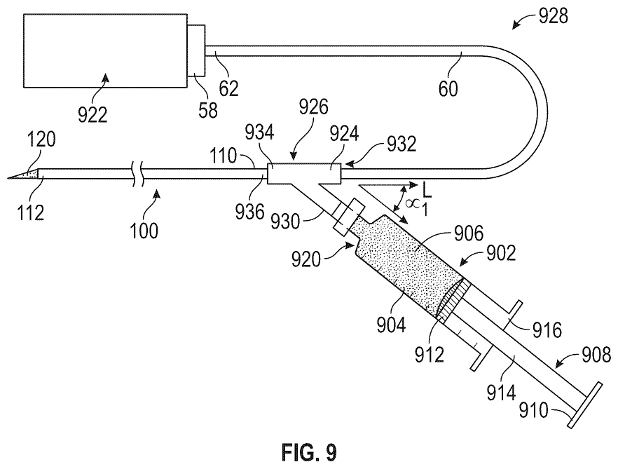

FIG. 9 illustrates an embodiment of a therapeutic delivery system 900 for use with a needle 100 including a coating as described herein. The therapeutic delivery system 900 includes a container 902 for holding a therapeutic agent 904 in a hollow volume 906 defined in the container 902. In the illustrated embodiment, the container 902 is a syringe including a plunger 908, slidable within the container 902. The plunger 908 includes a main body 914. At a first end of the main body 914 is a proximal head 910 that extends out of the proximal end 916 of the syringe container 902 and at a second end of the main body is a distal head 912 that is located within the hollow volume 906 and seals against the walls of the container 902. Forcing the proximal head 910 in a distal direction towards the distal end 920 of the container 902 causes the distal head 912 to force the therapeutic agent 904 out of the distal end 920 of the container 902. The distal end 920 of the container is connected to a proximal end 110 of a needle 100 including a coating as described herein by way of a connector 926.

In embodiments, the system 900 also includes a pressure source 922, such as a canister, for providing a pressurized fluid, such as a liquid or gas, to the system 900. The pressure source 922 is connected by way of a tube 928 to the connector 926. In the illustrated embodiment, the connector 926 is a y-connector including three legs. A first leg 924 at a proximal end 932 of the y-connector 926 is connected to the tube 928 from the pressure source, which provides a flow path for the pressurized fluid. The distal end 920 of the container 902 is connected to a second leg 930 the y-connector 926. The y-connector 926 joins the flow paths of the pressure source 922 and the distal end 920 of the container 902. A third leg 934 at the distal end 936 of the y-connector 926 is connected to the needle 100. As the therapeutic agent 904 is being delivered from the container 902, the pressurized fluid may carry the therapeutic agent 904 down the third leg 934 of the y-connector 926 and through the needle 100 where it is dispensed out of an opening 120 at the distal end 112 of the needle.

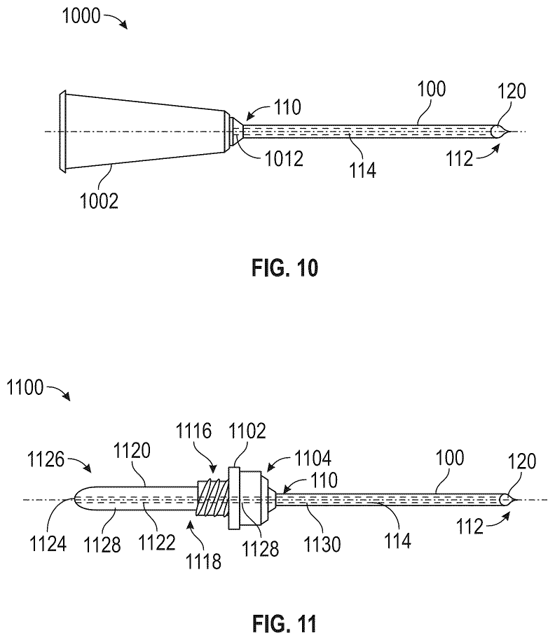

FIG. 10 illustrates a hypodermic needle 1000 that may be used for hypodermic injections. The hypodermic needle 1000 includes any one of the above described needles 100 including a coating. The hypodermic needle 1000 includes a connector 1002 at a proximal end 110 of the needle 100, which may be connected to a syringe, intravenous fluid dispenser, or other device. The needle 100 includes an opening 120 at the distal end 112 of the needle 100 and the needle 100 defines a bore 114 therein through which a therapeutic agent or intravenous fluid may flow. The connector 1002 also defines a bore 1012 therein that abuts bore 114 allowing fluid to flow through the connector 1002 and the bore 114.

FIG. 11 illustrates a blood collection needle 1100 used for drawing blood including a needle 100 with a coating as describe herein. The blood collection needle 1100 includes a hub 1102. Connected to the distal end 1104 of the hub 1102 is the proximal end 110 of the needle 100. The distal end 112 of the needle 100 includes an opening 120 and the needle 100 defines a bore 114. Connected to the proximal end 1116 of the hub 1102 is the distal end 1118 of a cannula 1120 which defines a lumen 1122 having an opening 1124 at a proximal end 1126 of the cannula 1120. The hub 1102 defines a passage 1128 therethrough connecting the flow path of the bore 114 to the flow path of the cannula lumen 1122. In embodiments, a blood collection vial is coupled at least one of the cannula 1120 and the hub 1102. This allows blood to be withdrawn through the needle 100, through the hub 1102, through the cannula 1120, and into the blood collection vial.

The needles and methods described herein offer a number of advantages. An advantage is the reduction in surface energy, enhancing the ability of the needle surface to repel water molecules. Similarly, the needle coatings may increase in water contact angle, increasing the hydrophobicity of the coated needle surface. An additional advantage of the present disclosure is a reduction in the accumulation of blood clots and other debris on surfaces of the needle, which may also reduce the likelihood of occlusions in openings. Another advantage, by reducing substances sticking to needle surfaces, is a smoother and relatively more consistent flow of fluids through or around the needle. A further advantage is an improvement in the efficient delivery of medications and relative increases in the accuracy and completeness of sample collection. Yet a further advantage is the reduction in the need for multiple insertions due to a reduction in obstructions.

An advantage also includes a potential reduction in the transfer of contaminants between different tissue or collection sites. An additional advantage is the relative simplicity of the immersion process and the ability of the mixture to reach both the external and interior surfaces of the needle. Another advantage is the compatibility of the coatings with sterilization procedures, including methods such as ethylene oxide sterilization and gamma sterilization. Yet another advantage is the compatibility of the coated needles with other devices and compositions used in performing procedures such as intravenous flush, delivery of saline solution, and delivery of many common medications. A further advantage is the relatively strong bond between the coatings and the metal cannula surfaces allowing the coating to adhere firmly to the substrate surface, preventing peeling or delamination and relatively increasing stability and longevity of the coating. Yet a further advantage is that the coating compositions do not damage the surface, the integrity, the functionality, and the performance of the needles.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A needle, comprising:
a metal cannula including an interior surface and an exterior surface; and
a coating disposed on at least one of the interior surface and the exterior surface, wherein
the coating is formed from a composition including a head group and a tail,
wherein the head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine, and
wherein the tail includes at least one of an alkyl including in the range of 4 carbons to 18 carbons, a cyclic group including 6 carbons, a polycyclic group including 10 carbons, and a fluoroalkyl including in the range of 4 carbons to 18 carbons.

2. The needle of claim 1, wherein the metal cannula includes at least one of stainless steel, titanium, nitinol, gold plated stainless steel, and cobalt-chromium.

3. The needle of claim 1, wherein the composition exhibits the formula (R—PO(OH)₂) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

4. The needle of claim 3, wherein the composition is octadecylphosphonic acid.

5. The needle of claim 3, wherein the composition is 3,3,4,4,5,5,6,6,7,7,8,8,8-tridecafluorooctylphosphonic acid.

6. The needle of claim 1, wherein the composition exhibits the formula (R—S—S—R) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

7. The needle of claim 1, wherein the composition exhibits the formula (R—SiX₃) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons, and X is at least one of the hydroxyl (—OH), the alkoxyl including in the range of 1 carbon to 6 carbons, and the halogen selected from at least one of chlorine, bromine, and iodine.

8. The needle of claim 1, wherein the composition exhibits the formula (R—SH) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

9. The needle of claim 1, wherein the composition exhibits the formula (R—S—R) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

10. A medical device, comprising:
a needle, including:
a metal cannula including an interior surface and an exterior surface, and
a coating disposed on at least one of the interior surface and the exterior surface, wherein
the coating is formed from a composition including a head group and a tail,
wherein the head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine, and
wherein the tail includes at least one of an alkyl including in the range of 4 carbons to 18 carbons, a cyclic group including 6 carbons, a polycyclic group including 10 carbons, and a fluoroalkyl including in the range of 4 carbons to 18 carbons.

11. A method of coating a needle, comprising:
mixing a composition with a first medium to form a mixture, wherein the composition includes a head group and a tail and the head group includes at least one of a thiol, a sulfide, a disulfide, a phosphonic acid, and a silane exhibiting the formula (—SiX₃), wherein X is at least one of a hydroxyl (—OH), an alkoxyl including in the range of 1 carbon to 6 carbons, and a halogen selected from at least one of chlorine, bromine, and iodine, and the tail includes at least one of an alkyl including in the range of 4 carbons to 18 carbons, a cyclic group including 6 carbons, a polycyclic group including 10 carbons, and a fluoroalkyl including in the range of 4 carbons to 18 carbons;
immersing a needle including a metal cannula in the mixture, wherein the metal cannula includes an interior surface and an exterior surface; and
forming a coating on at least one of the interior surface and the exterior surface of the metal cannula.

12. The method of claim 11, wherein the first medium includes at least one of ethanol, isopropanol, n-butanol, n-propanol, methanol, acetone, chloroform, and tetrahydrofuran.

13. The method of claim 11, wherein the composition is present in the first medium at a concentration on the order of 0.1 millimoles per liter to 10 millimoles per liter.

14. The method of claim 11, wherein the metal cannula is immersed in the mixture for a time period on the order of 5 minutes to 48 hours.

15. The method of claim 11, further comprising rinsing the metal cannula with a rinse after immersing the metal needle cannula, wherein the rinse includes at least one of ethanol, isopropanol, n-butanol, n-propanol, methanol, acetone, chloroform, and tetrahydrofuran.

16. The method of claim 11, further comprising drying the metal cannula including the coating at a temperature on the order of 100 degrees Celsius to 150 degrees Celsius.

17. The method of claim 11, wherein the metal cannula is at least one of stainless steel, titanium, nitinol, gold plated stainless steel, and cobalt-chromium.

18. The method of claim 11, wherein the composition exhibits the formula (R—PO(OH)₂) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.

19. The method of claim 11, wherein the composition exhibits the formula (R—SiX₃) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons and X is at least one of the hydroxyl (—OH), the alkoxyl including in the range of 1 carbon to 6 carbons, and the halogen selected from at least one of chlorine, bromine, and iodine.

20. The method of claim 11, wherein the composition exhibits the formula (R—SH) wherein R is at least one of the alkyl including in the range of 4 carbons to 18 carbons, the cyclic group including 6 carbons, the polycyclic group including 10 carbons, and the fluoroalkyl including in the range of 4 carbons to 18 carbons.
